(19) United States
(12) Patent Application Publication
(10) Pub. No.: US 2026/0327108 A1
(43) Pub. Date: Oct. 22, 2026

(54) WATER TREATMENT FACILITY OPERATION ASSISTANCE SYSTEM AND WATER TREATMENT FACILITY OPERATION ASSISTANCE METHOD

(71) Applicant: Mitsubishi Electric Corporation, Tokyo (JP)

(72) Inventors: Wataru YOSHIDA, Tokyo (JP); Yoshifumi HAYASHI, Tokyo (JP); Eiji IMAMURA, Tokyo (JP)

(73) Assignee: Mitsubishi Electric Corporation, Tokyo (JP)

(21) Appl. No.: 19/117,397

(22) PCT Filed: Dec. 6, 2022
(86) PCT No.: PCT/JP2022/044807

§ 371 Date: Apr. 1, 2025

(51) Int. Cl. C02F 3/00; C02F 1/00; C02F 3/12

(52) U.S. Cl. CPC C02F 3/006; C02F 3/12; C02F 2001/007; C02F 2209/006; C02F 2209/42

(57) ABSTRACT

This water treatment facility operation assistance system includes: an operating condition selection circuitry that selects at least one operating condition from a plurality of operating conditions; a control target value setting circuitry that sets a control target value corresponding to the operating condition selected by the operating condition selection circuitry; a simulation circuitry that, on the basis of a state quantity stored in a data storage circuitry and the control target value calculated by the control target value setting circuitry, carries out a simulation to predict at least one state quantity from a plurality of state quantities pertaining to the water treatment facility; and an output circuitry that outputs a simulation result from the simulation circuitry.

### TECHNICAL FIELD

The present disclosure relates to water treatment facility operation assistance system and water treatment facility operation assistance method.

### BACKGROUND ART

Conventionally, in a sewage treatment plant, an operation management system that performs water quality simulations based on state quantities has been provided. This system includes an input output unit for entering the operating conditions of a sewage treatment plant, a data storage unit for storing state quantities transmitted from the sewage treatment plant, a water quality prediction whit that incorporates a pre-installed water quality simulator and performs water quality simulations based on state quantities from the data storage unit and operating conditions from the input-output unit, and a parameter input unit for entering parameters of the water quality simulator into the water quality prediction unit. In this conventional system, the results of water quality simulation created by the water quality prediction unit are output to the input/output unit.

### CITATION LIST

### Patent Document

Patent Document 1: Japanese Laid-Open Patent Publication No. 2006-95440

### SUMMARY OF THE INVENTION

### Problem to be Solved by the Invention

For the operation of water treatment facilities, including sewage treatment plants, as described in the prior art, operation support technologies have been proposed to reduce reliance on operation monitoring based on know-how of skilled operators.

Operating conditions for water treatment facilities, including sewage treatment plants and water purification plants, are not limited to a single pattern. Operators must select the optimal conditions from various operational patterns, taking into account factors such as fluctuations in influent load, weather conditions, and impact of equipment shutdowns at the water treatment facilities.

Therefore, for an inexperienced user, selecting the optimum operating condition from various patterns of operating conditions becomes a difficult task.

The present disclosure aims to solve the aforementioned problems by providing an operation assistance system and an operation assistance method for a water treatment facility. This system enables even inexperienced users to select optimal operating conditions for water treatment facility operation.

### Means to Solve the Problem

The water treatment facility operation assistance system disclosed in the present disclosure is a water treatment facility operation assistance system for supporting the operation of a water treatment facility, comprising:
a data storage unit for storing state quantities indicating the status of the water treatment facility transmitted from the water treatment facility;
an operating condition selection unit for selecting at least one operating condition from among a plurality of operating conditions related to the operation of the water treatment facility;
a control target value setting unit for calculating a control target value corresponding to the operating condition selected by the operating condition selection unit;
a simulation unit for performing simulations to predict at least one state quantity from among a plurality of state quantities of the water treatment facility based on the state quantities stored in the data storage unit and the control target value calculated by the control target value setting unit; and
an output unit that displays the simulation results generated by the simulation unit.

The water treatment facility operation assistance system disclosed in the present disclosure is a water treatment facility operation assistance system for supporting the operation of a water treatment facility, comprising:
a data storage unit for storing state quantities indicating the status of the water treatment facility, transmitted from the water treatment facility;
a control target value input unit for inputting control target values related to the operation of the water treatment facility;
an operating condition display unit for displaying at least one operating condition from among a plurality of operating conditions corresponding to the control target values entered by the control target value input unit;
a simulation unit for performing a simulation related to the operation of the water treatment facility, based on the state quantities stored in the data storage unit and the control target values entered by the control target value input unit; and
an output unit for outputting the simulation results from the simulation unit.

The water treatment facility operation assistance method disclosed in the present disclosure is a water treatment facility operation assistance method for supporting the operation of a water treatment facility, comprising:
a data storage step for accumulating state quantities indicating the operational condition of the water treatment facility transmitted from the water treatment facility;
an operating condition selection step for selecting at least one operating condition from a plurality of operating conditions related to the operation of the water treatment facility;
a control target value setting step for calculating a control target value corresponding to the operating condition selected in the operating condition selection step;
a simulation step for performing a simulation to predict at least one of the state quantities of the water treatment facility based on the state quantities stored in the data storage step and the control target value calculated in the control target value setting step; and
an output step for outputting the simulation results from the simulation step.

The water treatment facility operation assistance method disclosed in the present disclosure is a water treatment facility operation assistance method for Supporting the operation of a water treatment facility, comprising:
a data storage step for storing state quantities indicating the status of the water treatment facility transmitted from the water treatment facility;
a control target value input step for accumulating State quantities indicating the condition of the water treatment facility transmitted from the facility;
a control target value input step for inputting control target values associated with the operation of the water treatment facility;
an operating condition display step for displaying at least one operating condition from a plurality of operating conditions corresponding to the control target values entered in the control target value input step;
a simulation step for performing a simulation related to the operation of the water treatment facility based on the state quantities stored in the data storage step and the control target values entered in the control target value input step; and
an output step for outputting the simulation results from the simulation step.

### Effect of the Invention

According to the operation assistance system and method disclosed in the present disclosure, even inexperienced users can select optimal operating conditions for the operation of a water treatment facility.

### BRIEF DESCRIPTION OF THE DRAWINGS

FIG. 1 a configuration diagram illustrating a water treatment facility and a water treatment facility operation assistance system according to embodiment 1.

FIG. 2 is a diagram illustrating an example screen of the operating condition selection unit according to embodiment 1.

FIG. 3 is a diagram illustrating an example of the setting of control target values in the control target value setting unit according to embodiment 1.

FIG. 4 is a diagram illustrating an example of water quality simulation results displayed on the output unit according to embodiment 1.

FIG. 5 is a diagram illustrating the operation flow of the water treatment facility operation assistance system according to embodiment 1.

FIG. 6 is a configuration diagram illustrating a water treatment facility and a water treatment facility operation assistance system according to embodiment 2.

FIG. 7 a configuration diagram illustrating a water treatment facility and a water treatment facility operation assistance system according to embodiment 3.

FIG. 8 is a configuration diagram illustrating a water treatment facility and a water treatment facility operation assistance system according to embodiment 4.

(FIG. 9 is a diagram illustrating an example of the hardware configuration of the water treatment facility operation assistance system according embodiments.

### DESCRIPTION OF EMBODIMENTS

Preferred embodiments of the present disclosure will be described in detail below with reference to the accompanying drawings. In the present specification and drawings, elements having substantially the same function are assigned the same reference numerals, and redundant explanations are omitted. It should be noted that the diagrams illustrating the system configuration and the shape of components merely depict schematic configurations and shapes of the system and components. The relative sizes and positions of the components shown in the drawings do not necessarily represent the exact size or positional relationships between the actual components.

In the following, a sewage treatment plant is used as an example of a water treatment facility for explanation purposes. However, the technology disclosed in the present disclosure applicable to water treatment facilities in general, including both water purification plants and sewage treatment plants.

### Embodiment 1

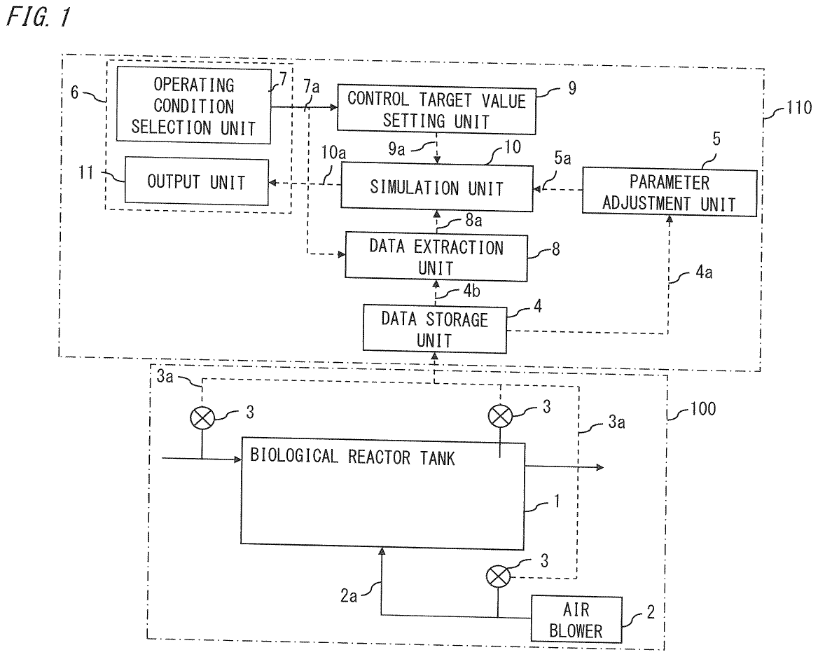

FIG. 1 is a configuration diagram of a water treatment facility and a wat treatment facility operation assistance system according to embodiment 1.

The water treatment facility 100 according to embodiment 1 includes a biological reactor tank 1, an air blower 2, and sensors 3. The water treatment facility operation assistance system 110 according to embodiment 1, includes a data storage unit 4, a parameter adjustment unit 5, an input-output unit 6, a data extraction unit 8, a control target value setting unit 9, and a simulation unit 10. The input-output unit 6 further includes an operating condition selection unit 7 and an output unit 11.

The details of the water treatment facility and the water treatment facility operation assistance system according to embodiment 1 will be described below.

The biological reactor tank 1 of the water treatment facility 100 contains activated sludge inside. Air supplied from the air blower 2 is supplied through the piping 2a into the biological reactor tank 1. The air blower 2 and piping 2a are not limited to a single set but can be arranged in multiples depending on the structure and size of the biological reactor tank 1.

The sensors 3 are sensors for measuring state quantities of the water treatment facility 100 and are installed at a plurality of locations within the water treatment facility 100. The sensors 3 include various types of sensors for measuring the state of the water treatment facility 100, such as flowmeters, airflow meters, thermometers, dissolved oxygen (DO) meters, PH meters, oxidation-reduction potential (ORP) meters, mixed liquor suspended solids (MLSS) meters, biochemical oxygen demand (BOD) meters, chemical oxygen demand (COD) meters, nicotinamide-adenine dinucleotide (NADH) meters, ammonia nitrogen concentration meters, total nitrogen concentration meters, total phosphorus concentration meters, polyphosphate phosphorus concentration meters, and chlorine concentration meters.

The data of a plurality of state quantities measured by the sensor 3 of the water treatment facility 100 is transmitted via the signal line 3 to the data storage unit 4 of the water treatment facility operation assistance system 110.

The data storage unit 4 of the water treatment facility operation assistance system 110 accumulates the data of a plurality of state quantities measured by the sensors 3, transmitted via the signal lines 3a. The larger the amount of accumulated data to be stored, the more desirable it becomes, and the system can store data ranging from periods of less than a day to several years.

The parameter adjustment unit 5 receives past state quantity data stored in the data storage unit 4 via signal line 4a and adjusts the parameters of the simulator built into the simulation unit 10 to reproduce the past state quantity data.

The input-output unit 6, which includes the operating condition selection unit 7 and the output unit 11, functions as a device for the input of operating conditions and the verification of simulation results. The input-output unit 6 includes, for example, a personal computer, a touch panel, a tablet, a smartphone, and the like.

In the operating condition selection unit 7, the user selects the desired conditions from among a plurality of preset operating conditions. The operating condition selection unit 7 enables the user to select conditions via the input output unit 6.

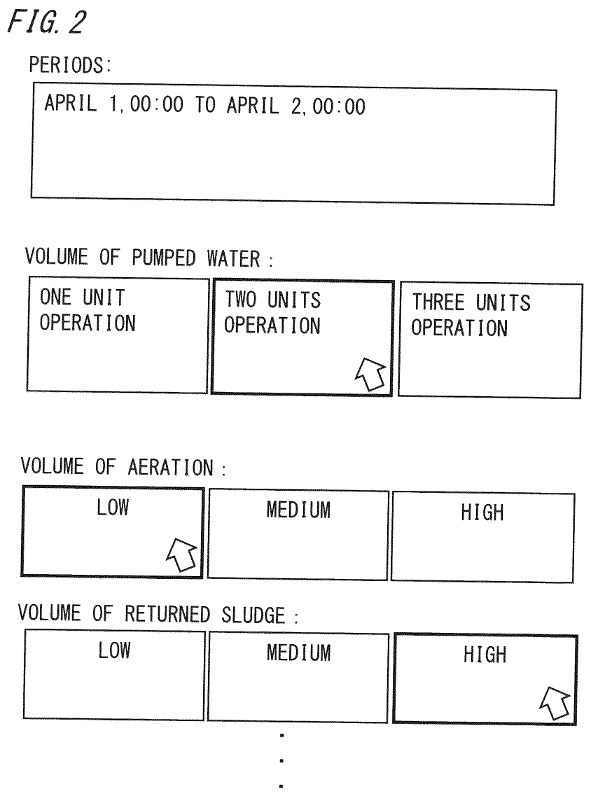

FIG. 2 shows an example of the screen of the operating condition selection unit 7 displayed on the input-output unit 6. In the operating condition selection unit 7, the user inputs the simulation period for the water treatment facility operation assistance system 110 according to embodiment 1.

In the example shown in FIG. 2, the period between April 1, 00:00, and April 2, 00:00, is entered as the simulation period.

In the operating condition selection unit 7, a plurality of operating conditions are preset for several parameters that serve as operation indicators for the water treatment facility 100. The user selects the desired operating conditions for simulation from among these presets.

In the example shown in FIG. 2, the operating indicators for the water treatment facility include parameters such as the volume of pumped water, the volume of aeration, and the volume of returned sludge, with three operating conditions set for each parameter in this example. The user selects the operating condition to be simulated for each parameter.

The operating indicators shown in FIG. 2 are merely examples. In general sewage treatment plants, typical operation indicators include the volume of pumped water, the volume of aeration, the volume of returned sludge, the volume of recirculated nitrification liquid, the volume of excess sludge withdrawal, the volume of coagulant dosing, the number of operating pumps, the number of operating blowers, bypass flow rate, and incinerator temperature. Given that operation indicators vary widely depending on the plant configuration, they can be modified, increased, or decreased as needed. For example, in a sewage treatment plant employing the Anaerobic-Anoxic-Oxic (A2O) process, the parameters available for selection in the operating condition selection unit 7 may include the volume of pumped water, aeration, returned sludge, and recirculated nitrification liquid.

In the data extraction unit 8, extraction is performed for data corresponding to the period desired by the user, as entered in the operating condition selection unit 7, from the data storage unit 4 via signal line 4b. If the period entered in the operating condition selection unit 7 is earlier than, or later than, the period stored in the data storage unit 4, the system may alternatively search for and create a dataset from the data storage unit 4 that matches the characteristics of the specified period (e.g., weekday/holiday, rainfall, season).

The operating conditions selected by the user in the operating condition selection unit 7 are transmitted to the control target value setting unit 9 via the signal line 7a, and the control target value setting unit 9 calculates the control target value required to achieve the operating conditions selected by the user and inputs it to the simulation unit 10.

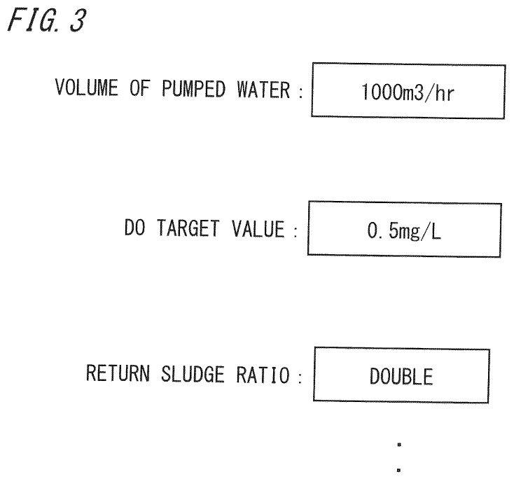

FIG. 3 shows an example of control target values set in the control target value setting unit 9. As shown in FIG. 2, if the volume of pumped water is selected as “two units operation,” the volume of aeration as “low,” and the volume of returned sludge as “high” in the operating condition selection unit 7, then the control target value setting unit 9 sets specific values for the volume of pumped water, DO target value, and returned sludge ratio as control target values corresponding to each operating condition. For example, if the volume of aeration is selected as “low” in the operating condition selection unit 7, a DO target value of 0.5 mg/L is set in the control target value setting unit 9. If the volume of aeration is selected as “medium,” a DO target value greater than 0.5 mg/L (e.g., 1 mg/L) is set.

As a method for converting the operating conditions selected in the operating condition selection unit 7 into specific control target values in the control target value setting unit 9, control target values corresponding to each operating condition may be pre-assigned based on the expertise of skilled operators. Alternatively, an Artificial Intelligence (AI) method may be used. For instance, by utilizing AI clustering techniques, AI can classify past state quantity data operated by skilled operators and automatically compute control target values corresponding to one/two/three-unit operation of pumped water, or low/medium/high levels of aeration or returned sludge, as shown in FIG. 3. Employing an AI-drived method enables even inexperienced users to automatically compute control targe values that approximate the operation of skilled operators when using the water treatment facility operation assistance system of this embodiment.

The correlation between the operating conditions and the corresponding control target values does not necessarily have to remain constant and, preferably, should be adjusted as appropriate according to seasonal variations, the operational status of the sewage treatment plant, and other factors.

FIG. 3 illustrates an example of a sewage treatment plant that employs DO control as the control method for aeration volume and ratio control relative to influent water volume as the control method for returned sludge volume. In addition to DO control, there are a wide variety of control methods for the aeration volume adopted by sewage treatment plants, such as constant aeration volume control, magnification control relative to the inflow water volume, and feedback control based on the values of state quantities in the biological reactor tank 1. Similarly, in the method of controlling the amount of returned sludge, there are methods such as constant flow rate control in addition to ratio control relative to the amount of inflow water. Furthermore, since the control methods for each operating indicator differ according to the sewage treatment plant, the items and values targeted for control also vary accordingly. For example, in a sewage treatment plant that adopts ratio control relative to influent water volume for aeration volume and constant flow control for returned sludge volume, the control target value for aeration volume in FIG. 3 would be the aeration volume ratio in place of the DO target value, and the control target value for returned sludge volume would be the returned sludge flow rate in place of the returned sludge ratio.

The simulation unit 10 is equipped with a built-in water quality prediction simulator and conducts water quality simulations using parameters transmitted from the parameter adjustment unit 5 via signal line 5a, state quantity data for the user-specified period transmitted from the data extraction unit 8 via signal line 8a, and control target values transmitted from the control target value setting unit 9 via signal line 9a.

In the example shown in FIG. 3, the simulation unit 10 performs a water quality simulation in accordance with the control target values set by the control target value setting unit 9, assuming the operation of the sewage treatment plant with a pumped water volume of 1000 m³/hr, a DO level of 0.5 mg/L in the biological reaction tank 1, and a returned sludge ratio of 2.

The water quality prediction simulator built into the simulation unit 10 simulates a plurality of water quality parameters, as specified by the user, in the biological reaction tank 1 or in the water discharged from the biological reaction tank 1. Representative water quality parameters include: DO, pH, ORP, MISS, BOD, COD, NADH, ammonia nitrogen concentration, total nitrogen concentration, total phosphorus concentration, polyphosphate phosphorus concentration, and chlorine concentration. The water quality prediction simulator can be implemented in any form capable of simulating the desired water quality parameters, including the activated sludge model proposed by the International Water Association (IWA), AI techniques such as machine learning, deep learning, and genetic algorithms, or formulas based on empirical rules.

The output unit 11 outputs the water quality simulation results transmitted from the simulation unit 10 via signal line 10a to a screen or the like.

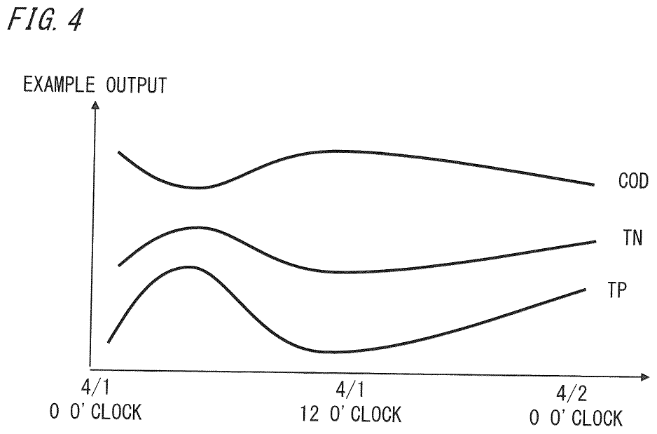

FIG. 4 shows an example of water quality simulation results displayed on the output unit 11. When COD, TN (Total Nitrogen), and TP (Total Phosphorus) of the water discharged from the biological reaction tank 1 are output to the output unit 11, the COD, TN, and TP data for the period specified by the user in the operating condition selection unit 7 are displayed as a trend graph on the screen. The output format of the data is not necessarily limited to a trend graph; it may also be displayed in the form of a data table, output as average values for the specified period, or in other formats specified by the user, such as summary tables or charts, among other options.

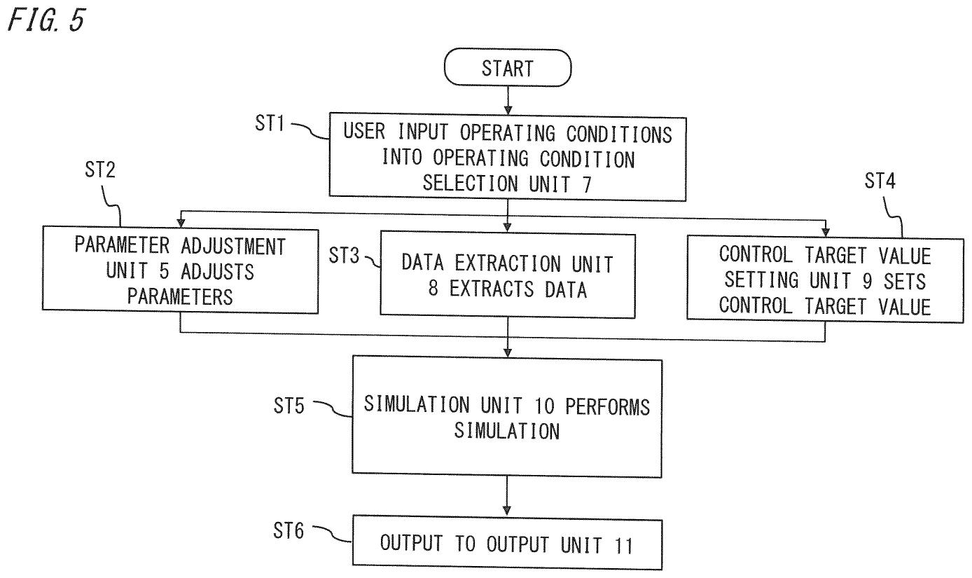

The operation flow of the water treatment facility operation assistance system 110 in embodiment 1 will be explained using FIG. 5.

The operation flow of the water treatment facility operation assistance system 110 includes six steps, from Step STI to Step ST6.

In Step ST1, the user inputs the operating conditions into the operating condition selection unit 7. Following this, the operation flow proceeds to Steps ST2 to ST4. Since Steps ST2 to S14 are independent steps, parallel operation is possible, as shown in FIG. 5, thereby reducing the operation time of the water treatment facility operation assistance system 110. However, it is not essential to perform steps ST2 to ST4 in parallel, and the same results can be obtained even if two or more of steps ST2 to ST4 are performed sequentially.

In Step ST2, the parameter adjustment unit 5 adjusts the parameters of the simulation unit 10 for reproducing the past state quantity data stored in the data storage unit 4.

In Step ST3, the data extraction unit 8 extracts data for the period specified by the user in the operating condition Selection unit 7 from the data storage unit 4.

In Step ST4, the control target value setting unit 9 calculates the control target values necessary to achieve the operating conditions selected by the user in the operating condition selection unit 7 and inputs the control target values into the simulation unit 10.

In Step ST5, the simulation unit 10 performs a water quality simulation based on parameters transmitted from the parameter adjustment unit 5, state quantity data transmitted from the data extraction unit 8, and control target values transmitted from the control target value setting unit 9.

In step ST6, the output unit 11 outputs the water quality simulation results.

After Steps ST1 to ST6 are completed, if the user enters different operating conditions in the operating condition selection unit 7, Steps ST1 to ST6 are repeated. This allows the user to repeatedly review the simulation results for a plurality of operating conditions, enabling them to observe the effects of changes in operating conditions on water quality and explore the optimal operating conditions.

With the above configuration and flow, even a user with limited experience in monitoring the operation of the water treatment facility 100 can simply select the desired conditions from among the preset operating conditions in the operating condition selection unit 7, allowing the control target value setting unit 9 to automatically set the control target values for the simulation. This makes it easy to perform water quality simulations for a plurality of operating conditions. Furthermore, since the user can view the water quality simulation results when operating conditions are changed, they can explore and determine the optimal operating conditions for the actual operation of the water treatment plant.

As described above, according t embodiment 1, a water treatment facility operation assistance syst supports the operation of a water treatment facility and comprises:
a data storage unit that stores state quantities indicating the state of the water treatment facility transmitted from the water treatment facility;
an operating condition selection unit that selects at least one operating condition from among a plurality of operating conditions related to the operation of the water treatment facility;
a control target value setting unit that calculates control target values corresponding to the operating conditions selected in the operating condition selection unit;
a simulation unit that performs simulations to predict at least one of the state quantities of the water treatment facility based on the state quantities stored in the data storage unit and the control target values calculated by the control target value setting unit; and
an output unit that outputs the simulation results of the simulation unit,
thereby enabling even users with limited experience in operating water treatment facilities to achieve optimal operating conditions.

A water treatment facility operation assistance method supports the operation of a water treatment facility and comprises:
a data storage step that stores state quantities indicating the state of the water treatment facility transmitted from the water treatment facility; an operating condition selection step that selects at least one operating condition from among a plurality of operating conditions related to the operation of the water treatment facility;
a control target value setting step that calculates control target values corresponding to the operating conditions selected in the operating condition selection step;
a simulation step that performs simulations to predict at least one of the state quantities of the water treatment facility based on the state quantities stored in the data storage step and the control target values calculated in the control target value setting step; and
an output step that outputs the simulation results of the simulation step,
thereby enabling even users with limited experience in operating water treatment facilities to achieve optimal operating conditions.

The operating condition selection unit is configured so that a plurality of operating conditions are set for each of the a plurality of items serving as operation indicators, to allow user selection of at least one operating condition for each item, thereby enabling even users with limited experience in water treatment facility operation to easily select the operating conditions.

The control target value setting unit calculates the control target values corresponding to the operating conditions based on either the predefined control target values assigned for each operating condition according to the knowledge of skilled operators or based on classifications of past state quantity data generated by AI (Artificial Intelligence) from operations by skilled operators, allowing the control target values to approximate those of skilled operators.

The water treatment facility includes a biological reaction tank that stores activated sludge, and the simulation unit is configured to simulate at least one of the water quality inside the biological reaction tank and the water quality of the water discharged from the biological reaction tank, thereby enabling even users with limited experience in water treatment facility operation to simulate water quality close to that achieved by skilled operators.

### Embodiment 2

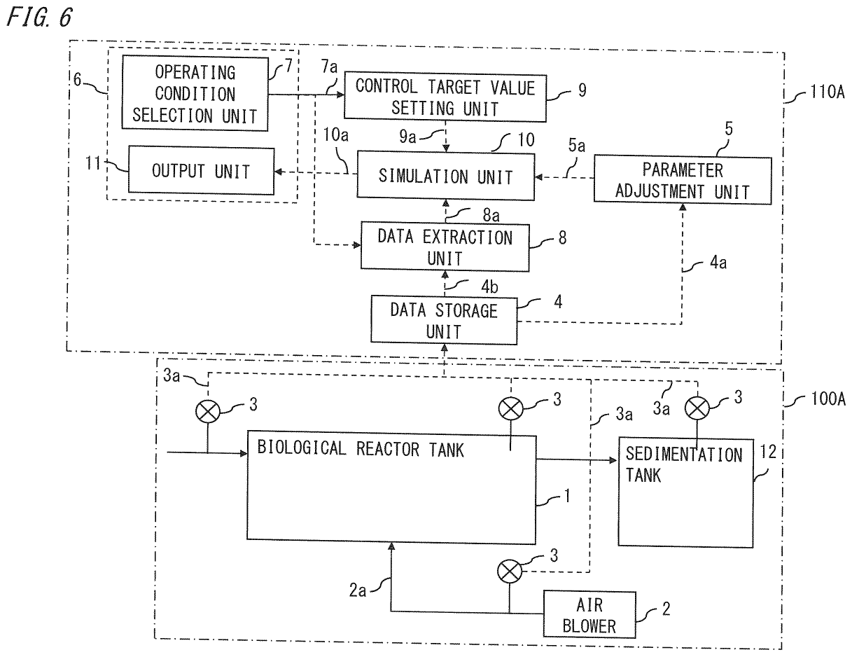

FIG. 6 illustrates the configuration of a water treatment facility and water treatment facility operation assistance system according to embodiment 2.

The water treatment facility 100A of embodiment 2 includes a biological reaction tank 1, an air blower 2, a sensor 3, and a sedimentation tank 12. The water treatment facility operation assistance system 110A of embodiment 2 includes a data storage unit 4, a parameter adjustment unit 5, an input/output unit 6, a data extraction unit 8, a control target value setting unit 9, a simulation unit 10, and an output unit 11, and the input/output unit 6 includes an operating condition selection unit 7 and an output unit 11.

The details of the water treatment facility and water treatment facility operation assistance system according to embodiment 2 will be explained below, focusing on the differences from embodiment 1.

The water treatment facility 100A of embodiment 2 includes a sedimentation tank 12 in addition to the configuration of embodiment 1. The sedimentation tank 12 receives water from the biological reaction tank 1, settling solids such as activated sludge to produce supernatant.

The sensors 3 are sensors for measuring the state of the water treatment facility and are installed at a plurality of locations in the water treatment facility 100A, including the sedimentation tank 12.

Various types of sensors correspond to sensor 3 for measuring the state of the water treatment facility 100A, including flow meters, airflow meters, thermometers, DO (Dissolved Oxygen) meters, ph meters, ORP (Oxidation-Reduction Potential) meters, MLSS (Mixed Liquor Suspended Solids) meters, BOD (Biochemical Oxygen Demand) meters, COD (Chemical Oxygen Demand) meters/NADH (Nicotinamide-Adenine Dinucleotide) meters, ammonia nitrogen concentration meters, total nitrogen concentration meters, total phosphorus concentration meters, polyphosphate phosphorus concentration meters, chlorine concentration meters, and sludge interface meters.

The water treatment facility operation assistance system 110A of embodiment 2, like embodiment 1, includes a data storage unit 4, a parameter adjustment unit 5, an input/output unit 6, an operating condition selection unit 7, a data extraction unit 8, a control target value setting unit 9, and a simulation unit 10, and the input/output unit 6 includes an operating condition selection unit 7 and an output unit 11.

The simulation unit 10 of embodiment 2 includes a built-in sludge interface height prediction simulator. The simulation unit 10 performs a prediction simulation of the sludge interface height in the sedimentation tank 12 based on parameters transmitted from the parameter adjustment unit 5 via signal line 5a, the state quantity data for the period specified by the user transmitted from the data extraction unit 8 via signal line 8a, and the control target values transmitted from the control target value setting unit 9 via signal line 9a.

Here, the sludge interface refers to the boundary between the settled solids and the supernatant liquid in the sedimentation tank 12, and the distance from the bottom of the sedimentation tank 12 to the sludge interface is the sludge interface height. The sludge interface height prediction simulator may take any form as long as it can simulate the sludge interface height in the settling tank 12, such as the Vesilind model, which is a physical model that describes the settling phenomenon of activated sludge in mathematical terms, AI such as machine learning, deep learning, or genetic algorithms, or mathematical formulas based on empirical rules.

The output unit 11 outputs the simulation results of the sludge interface height, transmitted from the simulation unit 10 via signal line 10a, to a screen or the like for review. Other components and the operation flow are the same as in embodiment 1.

In embodiment 2, the simulation unit 10 simulates the sludge interface height in the settling tank 12, and the sludge interface height is an important item to be monitored for stable operation of a sewage treatment facility. If the sludge interface height exceeds the height of the sedimentation tank 12, solids such as activated sludge may overflow into the effluent, causing a significantly deterioration in the quality of effluent. Furthermore, since activated sludge plays a role in purifying contaminants, the discharge of activated sludge from the sedimentation tank 12 may hinder further biological treatment. In particular, when the amount of water flowing into the biological reaction tank 1 increased significantly during rainy weather, the residence time required for solids to settle in the sedimentation tank 12 may not be secured, and the sludge interface height may rise.

For users with limited experience, monitoring operations under these conditions may often be challenging. However, in embodiment 2, the configuration and operation flow described above allow even users with limited experience in water treatment facility operation to easily perform sludge interface height simulations for a plurality of operating conditions by simply selecting desired conditions from among preset operating conditions in the operating condition selection unit 7, which automatically sets control target values in the control target value setting unit 9. Furthermore, the user can check the simulation results of the sludge interface height when the operating conditions are changed, allowing the user to search for and determine the optimal operating conditions and operate the actual water treatment plant.

As described above, according to embodiment 2, the same effects as those of embodiment 1 are achieved, and in addition, since the water treatment facility includes at least a sedimentation tank that settles solids of activated sludge and obtains a supernatant liquid, and the simulation unit performs a simulation of at least the sludge interface height in the sedimentation tank, even users with limited experience in water treatment facility operation can perform simulations of sludge interface heights similar to those achieved by skilled operators.

### Embodiment 3

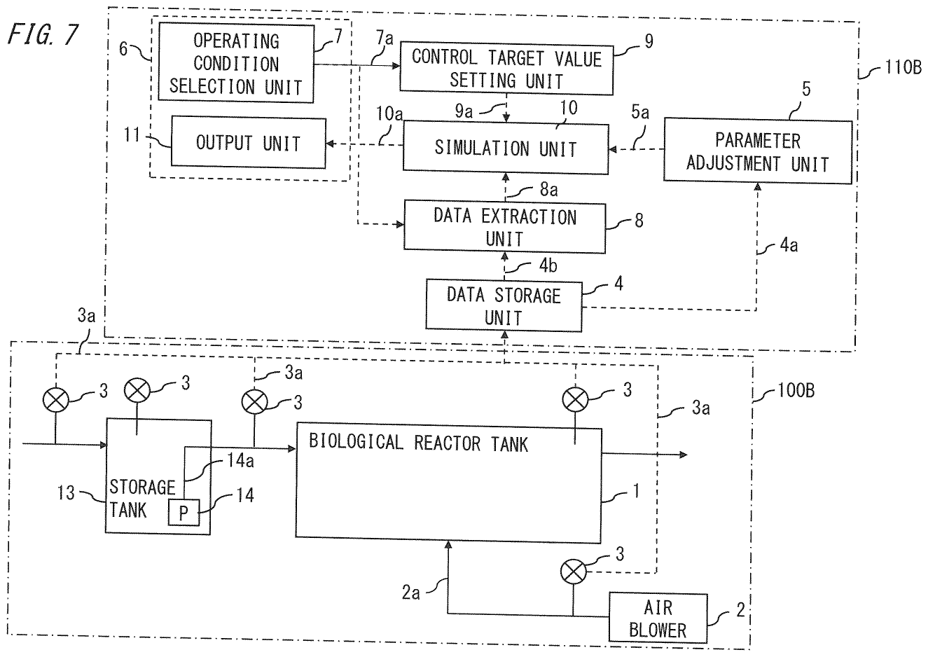

FIG. 7 shows the configuration of a water treatment facility and water treatment facility operation assistance system according to embodiment 3.

The water treatment facility 1008 of embodiment 3 includes a biological reaction tank 1, an air blower 2, a sensor 3, a storage unit 13, and a pump 14.

The water treatment facility operation assistance system 110B of embodiment 3 includes a data storage unit 4, a parameter adjustment unit 5, an input/output unit 6, a data extraction unit 8, a control target value setting unit 9, and a simulation unit 10, and the input/output unit 6 includes an operating condition selection unit 7 and an output unit 11.

The details of the water treatment facility and water treatment facility operation assistance system according to embodiment 3 will be explained below, focusing on the differences from embodiments 1 and 2.

The water treatment facility 100B of embodiment 3 includes a storage unit 13 and a pump 14, further including the configuration of embodiment 1.

The storage unit 13 temporarily stores wat before it flows into the biological reaction tank 1 in order to regulate the water inflow to the biological reaction tank 1. In general sewage treatment facilities, an inflow channel or a pump well serves as the storage unit 13, but as long as it has the aforementioned function, the storage unit 13 is not limited to an inflow channel or pump well.

The pump 14 is a pump that draws (lifts) the water stored in the storage unit 13 and allows it to flow into the biological reaction tank 1, with water from the storage unit 13 flowing into the biological reaction tank 1 via piping 14a.

The sensors 3 are sensors for measuring the state of the water treatment facility 100B and is installed in a plurality of locations within the water treatment facility 100B, including the piping that directs water into the storage unit 13 and the piping 14a that directs water from the storage unit 13 into the biological reaction tank 1. Various types of sensors correspond to sensor 3 for measuring the state of the water treatment facility 100B, including flow meters, airflow meters, thermometers, DO (Dissolved Oxygen) meters, pH meters, ORP (Oxidation-Reduction Potential) meters, MLSS (Mixed Liquor Suspended Solids) meters, BOD (Biochemical Oxygen Demand) meters, COD (Chemical Oxygen Demand) meters, NADH (Nicotinamide-Adenine Dinucleotide) meters, ammonia nitrogen concentration meters, total nitrogen concentration meters, total phosphorus concentration meters, polyphosphate phosphorus concentration meters, and chlorine concentration meters.

The water treatment facility operation assistance system 110B of embodiment 3, like embodiment 1, includes a data storage unit 4, a parameter adjustment unit 5, an input/output unit 6, an operating condition selection unit 7, a data extraction unit 8, a control target value setting unit 9, and a simulation unit 10, and the input/output unit 6 includes an operating condition selection unit 7 and an output unit 11.

The simulation unit 10 in embodiment 3 has a built-in water quality prediction simulator and a water level prediction simulator for the storage unit 13. The simulation unit 10 performs simulations of the water quality inside or discharged from the biological reaction tank 1 and the water level in the storage unit 13 based on parameters transmitted from the parameter adjustment unit 5 via signal line 5a, state quantity data for the period specified by the user transmitted from the data extraction unit 8 via signal line 8a, and control target values transmitted from the control target value setting unit 9 via signal line 9a.

The water level in th storage unit 13 is the distance from the bottom of the storage unit 13 to the water surface of the water stored in the storage unit 13, and fluctuates according to the balance between the amount of water flowing into the storage unit 13 and the amount of water pumped by pump 14.

The water level prediction simulator can take any form capable of simulating the water level in the storage unit 13, including a physical model describing the water balance in the storage unit 13, AI (Artificial Intelligence) such as machine learning, or empirical formula-based models.

The output unit 11 outputs the water quality simulation results and the storage unit 13 water level simulation results transmitted from the simulation unit 10 via signal line 10a to a screen or the like for user review. Other components and the operation flow are the same as in embodiment 1.

In embodiment 3, in addition to the water quality in the biological reaction tank 1 of the water discharged from the biological reaction tank 1, which is simulated by the simulation unit 10 in embodiment 1, a water level simulation of the storage unit 13 is also performed. By temporarily storing water in the storage unit 13, the amount of water flowing into the biological reaction tank 1 can be adjusted. Accordingly, the storage unit 13 has the effect of stabilizing the treatment process in the biological reaction tank 1.

However, when an unusually large amount of water, such as during rainfall, flows into the water treatment facility 100B, the amount of water flowing into the storage unit 13 significantly increases, causing the water level in the storage unit 13 to rise. If the water level in the storage unit 13 exceeds the height of the storage unit 13, water overflows from the storage unit 13, causing the water treatment facility 100B to become flooded with sewage. Therefore, is necessary to operate the water treatment facility 100B to keep the water level in the storage unit 13 from exceeding its height.

To reduce the water level in the storage unit 13, an effective approach is to increase the amount of water pumped to the biological reaction tank 1 by operating pump 14. However, increasing the inflow of water to the biological reaction tank 1 raises the pollution load within the tank, potentially leading to a deterioration in effluent quality.

Therefore, there is a trade-off relationship between the water level in the storage unit 13 and the quality of the water discharged from the biological reaction tank 1, and it is necessary to carry out operational monitoring of the entire water treatment facility 100B while taking both into consideration.

For users with limited experience, monitoring the water treatment facility 100B while considering such complex factors can be challenging. However, in embodiment 3, the configuration and operation flow described above allow even users with limited experience in operating the water treatment facility 100B to easily perform simulations of water quality and the water level in the storage unit 13 for a plurality of operating conditions. This is achieved by simply selecting desired conditions from among preset operating conditions in the operating condition selection unit 7, which automatically sets control target values in the control target value setting unit 9. Furthermore, since the user can check the water quality and the water level the storage unit 13 when the operating conditions are changed, the user can search for and determine the operating conditions that optimize both the water quality of the water discharged from the biological reaction tank 1 and the water level in the storage unit 13, and then operate the actual water treatment plant.

As described above, according to embodiment 3, the same effects as those of embodiments 1 and 2 are achieved, and in addition, since the water treatment facility includes a biological reaction tank that stores activated sludge and a storage tank that temporarily stores water flowing into the logical reaction tank, and the simulation unit simulates at least the water level in the storage tank, even users with limited experience in water treatment facility operation can perform simulations of storage tank water levels similar to those of skilled operators.

### Embodiment 4

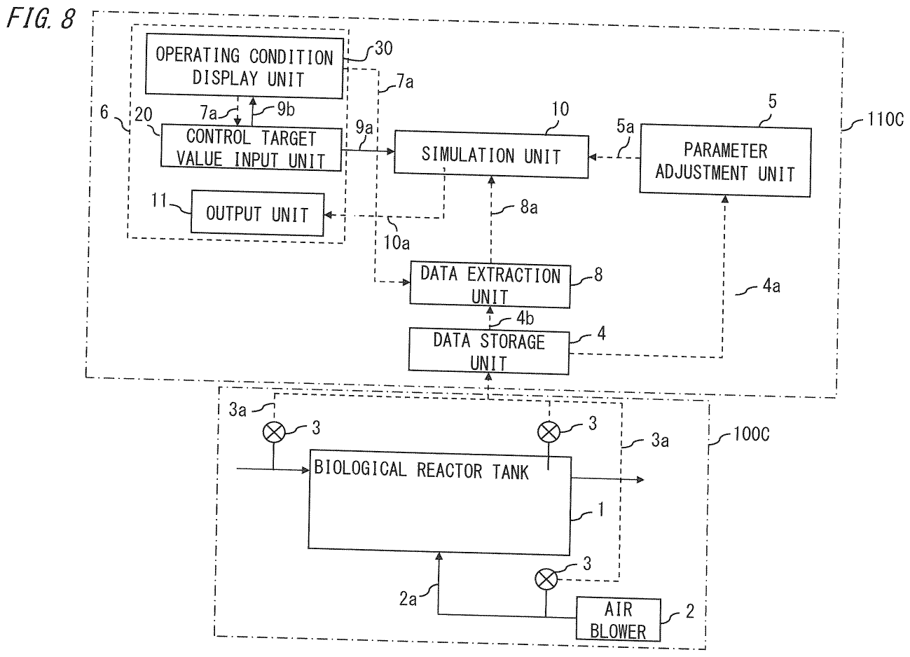

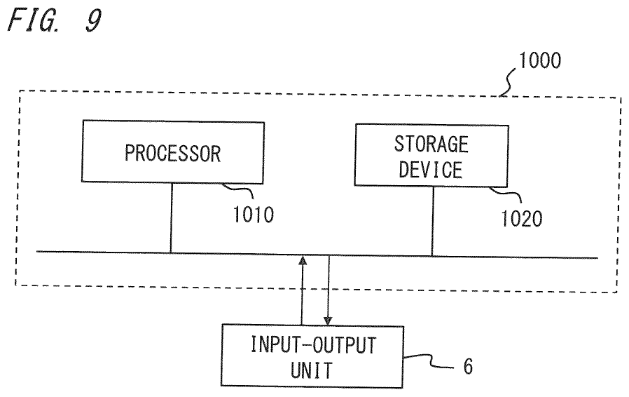

FIG. 8 shows the configuration of a water treatment facility and water treatment facility operation assistance system according to embodiment 4. The water treatment facility 100C of embodiment 4 includes a biological reaction tank 1, an air blower 2, and a sensor 3. The water treatment facility operation assistance system 110C of embodiment 4 includes a data storage unit 4, a parameter adjustment unit 5, an input/output unit 6, a data extraction unit 8, and a simulation unit 10, and the input/output unit 6 includes a control target value input unit 20, an operating condition display unit 30, and an output unit 11.

The details of the water treatment facility and water treatment facility operation assistance system according to embodiment 4 will be explained below, focusing on the differences from embodiments 1 through 3.

In embodiment 4, a control target value input unit 20 is provided in the input/output unit 6, and a user can directly input a control target value in the control target value input unit 20. The control target values input in the control target value input unit 20 are transmitted to the simulation unit 10 via signal line 9a and to the operating condition display unit 30 via signal line 9b.

When the control target value is transmitted to the operating condition display unit 30 via signal line 9b, the operating condition display unit 30 displays the operating conditions corresponding to the control target values specified in the control target value input unit 20. In the operating condition display unit 30, a plurality of operating conditions are set for each of a plurality of items that serve as operating indicators, and the respective operating conditions for each of the items that correspond to the control target values inputted in the control target value input unit 20 are displayed.

Using FIGS. 2 and 3 as examples, several operating indicators exist, such as the amount of pumped water, aeration, and returned sludge. For each indicator, a plurality of operating conditions are set: the amount of pumped water has “one-unit operation,” “two-unit operation,” and “three-unit operation”; aeration has “low,” “medium,” and “high”, and returned sludge has “low,” “medium,” and “high.” For instance, if a DO target value of 1 mg/L is specified in the control target value input unit 20, the aeration operating condition is displayed as “medium” instead of “low,” and the operating condition display unit 30 on the screen of the input/output unit 6 shows that the specified control target value corresponds to “medium” for aeration.

The simulation unit 10 performs a simulation of the water treatment facility operation based on the control target values entered in the control target value input unit 20. The simulation unit 10 incorporates at least one of the following simulators: a water quality prediction simulator, a sludge interface height prediction simulator, and a water level prediction simulator. Other components and the operation flow are the same as in embodiments 1 to 3.

With the above-described configuration, an experienced user with extensive knowledge about operation monitoring of water treatment facility can directly input the control target value to be set in the control target value input unit 20, instead of selecting pre-set operating conditions in the operating condition selection unit 7 of embodiments 1 to 3. This enables a more precise simulation of the operating conditions desired by the user. Furthermore, since the operating conditions corresponding to the control target values entered in the control target value input unit 20 are displayed in the operating condition display unit 30, even inexperienced users can easily understand what operating conditions are being simulated.

In embodiment 4, in case where use by an experienced user with extensive knowledge about operation monitoring water treatment plants is also assumed, the user is allowed to directly input the control target value to be set in the control target value input unit 20, rather than selecting pre-set operating conditions in the operating condition selection unit 7. In such a case, the operating conditions corresponding to the control target value inputted in the control target value input section 20 are displayed in the operating condition display section 30, so that an inexperienced user can learn under which operating conditions the simulation is being performed.

As described above, according to embodiment 4, a water treatment facility operation assistance system is provided to support the operation of a water treatment facility. The system comprises:
a data storage unit that accumulates state quantities indicating the condition of the water treatment facility, as transmitted from the facility;
a control target value input unit for entering control target values related to the facility's operation;
an operating condition display unit that shows at least one of the plurality of operating conditions corresponding to the control target values specified in the control target value input unit;
a simulation unit that performs a simulation of the facility's operation using the state quantities stored in the data storage unit along with the control target values specified in the control target value input unit; and
an output unit that outputs the simulation results from the simulation unit, enabling even inexperienced users to understand and optimize operating conditions for the water treatment facility.

The operating condition display has operating conditions set for each of the various operating indicators, and each of the operating conditions corresponding to each of these indicators for the control target values entered in the control target value input unit is displayed, allowing even inexperienced users to understand optimal operating conditions for the water treatment facility.

Additionally, a method for supporting the operation of a water treatment facility is provided, comprising:
a data storage step that accumulates state quantities transmitted from the facility, indicating its operational condition; a control target value input step for entering control target values associated with the operation of the facility;
an operating condition display step that displays at least one of the operating conditions corresponding to the specified control target values;
a simulation step that performs simulations related to facility operation based on the state quantities stored in the data storage step and the control target values entered in the control target value input step; and
an output step that displays the results from the simulation step, enabling even inexperienced users to understand and apply optimal operating conditions for the water treatment facility.

Furthermore, the water treatment facility operation assistance system of embodiment 4 may be used in combination with the water treatment facility operation assistance systems of embodiments 1 to 3.

Specifically, water treatment facility operation assistance system is provided, comprising:
a data storage unit that accumulates state quantities transmitted from the water treatment facility indicating its operational state;
an operating condition selection unit that selects at least one operating condition from among a plurality of conditions related to the operation of the facility;
control target value setting unit that calculates control target values corresponding to the operating conditions selected by the operating condition selection unit;
a simulation unit that, based on the state quantities stored in the data storage unit and the control target values calculated by the control target value setting unit, performs a simulation to predict at least one of the state quantities of the water treatment facility; and
an output unit that displays the simulation results generated by the simulation unit. Additionally, the system includes a control target value input unit that allows users to input control target values related to facility operation, and an operating condition display unit that displays at least one operating condition from among a plurality of conditions corresponding to the control target values entered in the control target value input unit.

The simulation unit may be configured to perform simulations related to facility operation based on the state quantities stored in the data storage unit and the control target values entered in the control target value input unit.

FIG. 9 shows example of the hardware configuration of water treatment facility operation assistance systems 110, 110A, 110B, and 110C according to embodiments 1 to 4.

As shown in FIG. 9, the water treatment facility operation assistance systems 110, 110A, 110B, and 110C include a processing device 1000 and an input/output unit 6. The processing device 1000 includes a processor 1010 and a storage device 1020. The storage device 1020 includes volatile memory such as random access memory and non-volatile auxiliary storage such as flash memory, though a hard disk may be used instead of flash memory.

The processor 1010 executes programs loaded from the storage device 1020, receiving programs from the auxiliary storage device through volatile memory. The processor 1010 may output calculation results and other data to volatile memory within the storage device 1020 or store data to the auxiliary storage device through volatile memory.

The processor 1010 performs the calculations for the parameter adjustment unit 5, data extraction unit 8, control target value setting unit 9, and simulation unit 10 described in previous embodiments. The storage device 1020 includes the data storage unit 4.

In addition, in the above-described embodiment, a sewage treatment facility is used as an example of a water treatment facility, but the technology disclosed in the present application can be applied to water treatment facilities in general, including water purification facilities and sewage treatment facilities.

For instance, when applied to a water purification facility, the water facility may include a receiving well, a coagulation tank, a sedimentation tank, a filtration tank, and the like. The operating indicators in the operating condition selection unit and operating condition display unit of the water treatment facility operation assistance system may include items such as sodium hypochlorite injection amount, ozone diffusion volume, and coagulant injection amount.

Although the disclosure is described above in terms of various exemplary embodiments and implementations, it should be understood that the various features, aspects, and functionality described in one or more of the individual embodiments are not limited in their applicability the particular embodiment with which they are described, but they can be applied, alone or in various combinations to one or more of the embodiments of the disclosure.

It is therefore understood that numerous modifications which have not been exemplified can be devised without departing from the scope of the present disclosure. For example, at least one of the constituent components may be modified, added, or eliminated. At least one of the constituent components mentioned in at least one of the preferred embodiments may be selected and combined with the constituent components mentioned in another preferred embodiment.

### DESCRIPTION OF THE REFERENCE CHARACTERS

1 Biological reactor tank
2 Air blower
3 Sensor
4 Data storage unit
5 Parameter adjustment unit
6 Input-output unit
7 Operating condition selection unit
8 Data extraction unit
9 Control target value setting unit
10 Simulation unit
11 Output unit
12 Sedimentation tank
13 Storage tank
20 Control target value input unit
30 Operating condition display unit
100, 100A, 100B, 100C Water treatment facility
110, 110A, 110B, 110C Operation assistance system

1. A water treatment facility operation assistance system for supporting the operation of a water treatment facility, comprising:
a data storage circuitry that accumulates state quantities indicating the operational condition of the water treatment facility transmitted from the water treatment facility;
a period input circuitry for inputting a period for which a simulation is to be performed;
an operating condition selection circuitry that selects at least one operating condition from a plurality of operating conditions related to the operation of the water treatment facility;
a control target value setting circuitry that calculates a control target value corresponding to the operating condition selected by the operating condition selection circuitry;
a simulation circuitry that, based on the state quantities stored in the data storage circuitry and the control target value calculated by the control target value setting circuitry, performs a simulation to predict at least one of the state quantities of the water treatment facility; and
an output circuitry that outputs transition information indicating a transition of the state quantity during the period inputted to the period input circuitry as the simulation results from the simulation circuitry.

2. The water treatment facility operation assistance system according to claim 1, wherein
the operating condition selection circuitry is configured to set a plurality of operating conditions for each of a plurality of items that serve as operating indicators, and to select at least one operating condition from the plurality of operating conditions for each of the items.

3. The water treatment facility operation assistance system according to claim 1, wherein
the control target value setting circuitry calculates the control target value corresponding to the operating condition based on a value previously assigned to each of the operating conditions based on the knowledge of an experienced operator, or calculates the control target value based on past state quantity data from operation by an experienced operator classified by AI (Artificial Intelligence).

4. A water treatment facility operation assistance system for supporting the operation of a water treatment facility, comprising:
a data storage circuitry that accumulates state quantities indicating the operational condition of the water treatment facility transmitted from the water treatment facility;
a period input circuitry for inputting a period for which a simulation is to be performed;
a control target value input circuitry for inputting control target values related to the operation of the water treatment facility;
an operating condition display circuitry that displays at least one operating condition among a plurality of operating conditions corresponding to the control target values inputted through the control target value input circuitry;
a simulation circuitry that performs a simulation related to the operation of the water treatment facility based on the state quantities stored in the data storage circuitry and the control target values entered in the control target value input circuitry; and
an output circuitry that outputs transition information indicating a transition of the state quantity during the period inputted to the period input circuitry as the simulation results from the simulation circuitry.

5. The water treatment facility operation assistance system according to claim 4, wherein
the operating condition display circuitry sets a plurality of operating conditions for each of a plurality of items that serve as operating indicators, and displays the respective operating conditions for each of the items that correspond to the control target values inputted in the control target value input circuitry.

6. The water treatment facility operation assistance system according to claim 1, wherein
the water treatment facility includes a biological reaction tank that stores activated sludge, and the simulation circuitry performs a simulation of at least one of the water quality in the biological reaction tank and the water quality discharged from the biological reaction tank.

7. The water treatment facility operation assistance system according to claim 1, wherein
the water treatment facility includes at least a sedimentation tank that settles solids of activated sludge and obtains a supernatant liquid, and the simulation circuitry performs a simulation of at least the sludge interface height in the sedimentation tank.

8. The water treatment facility operation assistance system according to claim 1, wherein
the water treatment facility includes a biological reaction tank that stores activated sludge and a storage tank that temporarily stores water flowing into the biological reaction tank, and the simulation circuitry performs a simulation of at least the water level in the storage tank.

9. A water treatment facility operation assistance method for supporting the operation of a water treatment facility, comprising:
accumulating state quantities indicating the operational condition of the water treatment facility transmitted from the water treatment facility;
inputting a period for which a simulation is to be performed;
selecting at least one operating condition from a plurality of operating conditions related to the operation of the water treatment facility;
calculating a control target value corresponding to the operating condition selected;
performing a simulation to predict at least one of the state quantities of the water treatment facility based on the state quantities stored and the control target value calculated; and
outputting transition information indicating a transition of the state quantity during the period inputted as the simulation results.

10. A water treatment facility operation assistance method for supporting the operation of a water treatment facility, comprising:
storing state quantities indicating the status of the water treatment facility transmitted from the water treatment facility;
inputting a period for which a simulation is to be performed;
accumulating state quantities indicating the condition of the water treatment facility transmitted from the facility;
inputting control target values associated with the operation of the water treatment facility;
displaying at least one operating condition from a plurality of operating conditions corresponding to the control target values entered;
performing a simulation related to the operation of the water treatment facility based on the state quantities stored and the control target values entered; and
outputting transition information indicating a transition of the state quantity during the period inputted as the simulation results.

11. The water treatment facility operation assistance system according to claim 2, wherein
the control target value setting circuitry calculates the control target value corresponding to the operating condition based on a value previously assigned to each of the operating conditions based on the knowledge of an experienced operator, or calculates the control target value based on past state quantity data from operation by an experienced operator classified by AI (Artificial Intelligence).

12. The water treatment facility operation assistance system according to claim 2, wherein
the water treatment facility includes a biological reaction tank that stores activated sludge, and the simulation circuitry performs a simulation of at least one of the water quality in the biological reaction tank and the water quality discharged from the biological reaction tank.

13. The water treatment facility operation assistance system according to claim 4, wherein
the water treatment facility includes a biological reaction tank that stores activated sludge, and the simulation circuitry performs a simulation of at least one of the water quality in the biological reaction tank and the water quality discharged from the biological reaction tank.

14. The water treatment facility operation assistance system according to claim 5, wherein
the water treatment facility includes a biological reaction tank that stores activated sludge, and the simulation circuitry performs a simulation of at least one of the water quality in the biological reaction tank and the water quality discharged from the biological reaction tank.

15. The water treatment facility operation assistance system according to claim 2, wherein
the water treatment facility includes at least a sedimentation tank that settles solids of activated sludge and obtains a supernatant liquid, and the simulation circuitry performs a simulation of at least the sludge interface height in the sedimentation tank.

16. The water treatment facility operation assistance system according to claim 4, wherein
the water treatment facility includes at least a sedimentation tank that settles solids of activated sludge and obtains a supernatant liquid, and the simulation circuitry performs a simulation of at least the sludge interface height in the sedimentation tank.

17. The water treatment facility operation assistance system according to claim 5, wherein
the water treatment facility includes at least a sedimentation tank that settles solids of activated sludge and obtains a supernatant liquid, and the simulation circuitry performs a simulation of at least the sludge interface height in the sedimentation tank.

18. The water treatment facility operation assistance system according to claim 2, wherein
the water treatment facility includes a biological reaction tank that stores activated sludge and a storage tank that temporarily stores water flowing into the biological reaction tank, and the simulation circuitry performs a simulation of at least the water level in the storage tank.

19. The water treatment facility operation assistance system according to claim 4, wherein
the water treatment facility includes a biological reaction tank that stores activated sludge and a storage tank that temporarily stores water flowing into the biological reaction tank, and the simulation circuitry performs a simulation of at least the water level in the storage tank.

20. The water treatment facility operation assistance system according to claim 5, wherein
the water treatment facility includes a biological reaction tank that stores activated sludge and a storage tank that temporarily stores water flowing into the biological reaction tank, and the simulation circuitry performs a simulation of at least the water level in the storage tank.
